(19) United States
(12) Patent Application Publication
(10) Pub. No.: US 2009/0123996 A1
(43) Pub. Date: May 14, 2009

(54) Vitrification Device with Shape Memory Seal

(76) Inventor: Milton Chin, Trumbull, CT (US)

Correspondence Address: Markets, Patents & Alliances LLC, 30 Glen Terrace, Stamford, CT, 06906-1404, US

(21) Appl. No.: 12/267,794

(22) Filed: Nov. 10, 2008

Related U.S. Application Data
(63) Continuation-in-part of application No. 12/267,708, filed on Nov. 10, 2008.
(60) Provisional application No. 60/987,110, filed on Nov. 12, 2007.

(51) Int. Cl. C12M 1/00

(52) U.S. Cl. 435/307.1

(57) ABSTRACT

A closing device to create a seal in a cryocontainer for a biological specimen utilizes the temperature-induced phase transformation of shape memory materials to cause an actuator to toggle between a sealed and unsealed state. The temperature inducement occurs naturally within the normal temperature changes that occur during cryogenic vitrification of biological specimens.

### Cross-Reference to Related Applications

This application is a continuation in part of U.S. nonprovisional patent application Ser. No. 12/267,708 filed on Nov. 10, 2008 entitled “Shape-Shifting Vitrification Device”. Said nonprovisional application is incorporated herein by reference.

Said nonprovisional patent application Ser. No. 12/267,708, in turn, claims priority to U.S. provisional patent application entitled “Shape Memory Vitrification Cryocontainer”, Ser. No. 60/987,110 filed on Nov. 12, 2007. Said provisional application is incorporated herein by reference.

### TECHNICAL FIELD

This invention is in the field of devices for the cryopreservation of biological specimens.

### BACKGROUND

Cryopreservation is practiced in the life sciences for the purpose of halting biological activity in valuable cell(s) for an extended period of time. One factor in the success of cryopreservation is reducing or eliminating the deleterious effect of ice crystal formation. Sophisticated methods are needed to thwart the natural tendency of water to freeze into ice during cryopreservation.

### Cryopreservation

One method of minimizing ice crystal formation is called “slow-freeze.” The initial step in slow-freeze is to dehydrate a cell or cells with an aqueous solution (“slow-freeze media”) containing permeating and non-permeating cryoprotectants (“CPA”). The cell or cells, together with a small quantity of slow-freeze media, comprise the “biological specimen.” The biological specimen is then placed in a suitable cryocontainer, i.e. a container suitable for use at cryogenic temperatures. As used herein, “cryogenic temperatures” means temperatures colder than −80° C. Slow-freeze cryopreservation entails chilling the biological specimen from room temperature to its ultimate cryogenic storage temperature that is typically −196° C., the atmospheric boiling point of liquid nitrogen (“LN2”). For a portion of this temperature range, from approximately −6° C. down to −30° C., the chilling rate is precisely controlled to 0.1-0.3° C./minute by a programmable freezer. Chilling from −30° C. to −196° C. is achieved by plunging the cryocontainer in LN2. Slow-freeze processes take 2-3 hours to complete, hence the name. By this process, ice crystals do form in the CPA surrounding the cell or cells, and minimally within the cell or cells. Slow-freeze is effective in cells with low water content such as embryos and sperm, but does not perform as well in high water content cells such as oocytes and blastocysts. This deficiency, high equipment cost, and the high consumption of time have lead to the development of an alternative cryopreservation method called vitrification.

### Vitrification

Vitrification differs from slow-freeze in that it seeks to avoid the formation of cell-damaging ice altogether. Similar to slow-freeze, the first step in vitrification is to dehydrate the cell or cells as much as possible using CPA containing fluids called “vitrification media.” The biological specimen (same definition as slow-freeze) is then rapidly chilled by immersion in a cryogenic fluid such as LN2. With a proper combination of chilling speed and CPA concentration, intracellular water will attain a solid, innocuous, glassy (vitreous) state rather than an orderly, damaging, crystalline ice state. Vitrification can be described as a rapid increase in fluid viscosity that traps the water molecules in a random orientation. Vitrification media, however, contain higher levels of CPA than slow-freeze media and are toxic to cells except in the vitreous state. Therefore, the time exposure of cells to vitrification media during dehydration and thawing (called “warming” since ice is not formed) must be carefully controlled to avoid cellular injury. The end point of vitrification and slow-freeze is the same: long term storage in a cryogen such as LN2.

If a chilling speed of 10⁶° C./minute were possible, vitrification could be achieved with no cryoprotectants at all. Extremely toxic vitrification media, with 60% w/w CPA concentration, can be vitrified with ordinary chilling speed. Commercial vitrification media have CPA formulations and minimum enabling chilling speeds between these boundaries. The inverse relationship between CPA concentration and minimum enabling chilling speed is well known. The key to minimizing the toxic effects of vitrification media is to minimize its CPA concentration. Therefore, it is desirable to chill quickly; the faster the better. Given this, a natural initial discovery in this field was to directly plunge the biological specimen into LN2 to achieve rapid chilling. Carrier devices to enable direct plunge were created to facilitate and control this process. Examples are: electron microscopy grids, open pulled straws, Cryoloop™, nylon mesh, and Cryotop. Cryoloop is a trademark of Hampton Research. These devices are classified as “open carriers” in that the biological specimen is in direct contact with the chilling cryogen, typically LN2. Open carriers also enabled rapid warming of the biological specimen.

LN2, however, is not aseptic. It may contain bacterial and fungal species, which are viable upon warming. Furthermore, it has been reported that vitrified cells held in long term storage in LN2 could be infected by viral pathogens artificially placed in said LN2. Hence, there is the potential for infection of biological samples vitrified in open carriers.

The potential of infection has lead to the development of closed cryocontainers where the biological sample is placed in a cryocontainer and sealed before chilling in LN2. The cryocontainer also serves as a storage device to isolate it from pathogen-containing cryogen during long-term storage.

### Limitations of Current Cryocontainers for Vitrification

U.S. Pat. No. 7,316,896, “Egg freezing and storing tool and method”, ('896 Device) describes a closed cryocontainer for vitrification. This device comprises a fine plastic tube (nominally 0.25 mm OD and a wall thickness of 0.02 mm). A typical biological specimen will contain a human oocyte having an OD of 0.125 mm. It is dehydrated with vitrification media and then drawn into the tube. Then both ends of the tube are heat-sealed with a thermal sealing device to create an aseptic container. Because one of the heat seals is created very close to the biological specimen, there are concerns that the heat will injure the cell.

U.S. Patent Application 2008/0220507, “Kit for Packaging Predetermined Volume of Substance to be Preserved by Cryogenic Vitrification”, ('507 Device) describes a tube-within-a-tube closed cryocontainer concept. Both tubes are fabricated from plastic. The inner tube is modified to create a channel at one end upon which the biological specimen is placed. The loaded inner tube is then placed within the outer tube. The outer tube is then heat-sealed at the loading end to create an aseptic cryocontainer.

Heat-sealing requires a costly sealing device capable of fusing the plastic of a vitrification cryocontainer. It also adds another step in a process that requires speed for safe execution. There is a need for a sealing concept overcomes these obstacles.

### SUMMARY OF THE INVENTION

The Summary of the Invention is provided as a guide to understanding the invention. It does not necessarily describe the most generic embodiment of the invention or all species of the invention disclosed herein.

### Improved Closed Vitrification Cryocontainer

The present invention comprises a closed vitrification cryocontainer comprising a closure device to seal and unseal the cryocontainer. This closure device utilizes the unique material characteristics of shape memory materials. Shape memory materials exist in two crystallographic structures: high temperature austenite and low temperature martensite. The austenite phase is characterized by stiffness and superelastic properties. The martensite phase is soft and malleable. The shape of an object in its austenite phase is referred to as the “memorized shape.” If a shape memory material is cooled from its austenite phase to its martensite phase and then deformed, it will return to its austenite shape when it is heated back into its austenite phase. Shuttling between these two phases enables design options that can advantageously be incorporated into the present invention.

### BRIEF DESCRIPTION OF DRAWINGS

FIG. 1 is a diagram that shows the relationship between the crystallographic state of a shape memory material (exhibiting one-way shape memory) and temperature.

FIG. 2 is a diagram that shows the relationship between the crystallographic state of a shape memory material (exhibiting two-way shape memory) and temperature.

FIG. 3 illustrates an actuator made by joining a shape memory material and a non-shape memory bias spring.

FIG. 4 illustrates a two-way shape memory spring.

FIG. 5 illustrates a roll pin actuator made by joining a shape memory material and a non-shape memory roll pin.

FIG. 6 illustrates a shape memory closing device.

FIG. 7 illustrates features of the shuttle, sheath, and cryocontainer.

FIG. 8 shows features of an assembled cryocontainer.

FIG. 9 illustrates a stopper that incorporates shape memory roll pins.

FIG. 10 describes the nitinol transformation temperatures suitable for this invention.

### DETAILED DESCRIPTION

The following detailed description discloses various embodiments and features of the invention. These embodiments and features are meant to be exemplary and not limiting.

As used herein, except for temperature and unless specifically indicated otherwise, the term “about” means within ±20% of a given value. With respect to temperature, “about” means within ±2° C.

A variety of biological cells can be aseptically cryopreserved (e.g. vitrified) using the present invention. One category of cells is mammalian developmental cells such as sperm, oocytes, embryos, morulae, blastocysts, and other early embryonic cells. These cells are routinely cryopreserved during assisted reproduction procedures. Another category of cells is stem cells that are used in regenerative therapies. The broadest category is any cell that can be vitrified using a vitrification media that aligns with the available chilling speed of this invention.

### Shape Memory Effect

The shape memory effect exists in alloys of certain metals such as Ag—Cd, Au—Cd, Cu—Al—Ni, Cu—Zn—Al, Cu—Zn—Si, Cu—Zn—Sn, Cu—Sn, Cu—Zn, Fe—Pt, Fe—Mn—Si, In—Ti, Mn—Cu, Mn—Si, Ni—Ti, Ni—Al, and others. Of this group, alloys of Ni—Ti are the most commercially prevalent variant and are referred to as nitinol. This invention can be implemented by a wide variety of shape memory alloys. The specific alloy to be use can be selected by those skilled in the art. To facilitate the understanding of this invention, the properties of nitinol as the shape memory material will be used in this Description to illustrate the features of this invention.

The shape memory effect is a phenomenon in which an object can exist in two different crystallographic states. The object in the first, higher temperature state is rigid with a unique defined shape. Upon cooling, this object changes to a readily deformable state. The object can be made to lose its deformability and metamorphose back to its unique defined shape by heating the material. Materials science teaches us that shuttling between these physical states is a phenomenon caused by a temperature-induced phase change of the material.

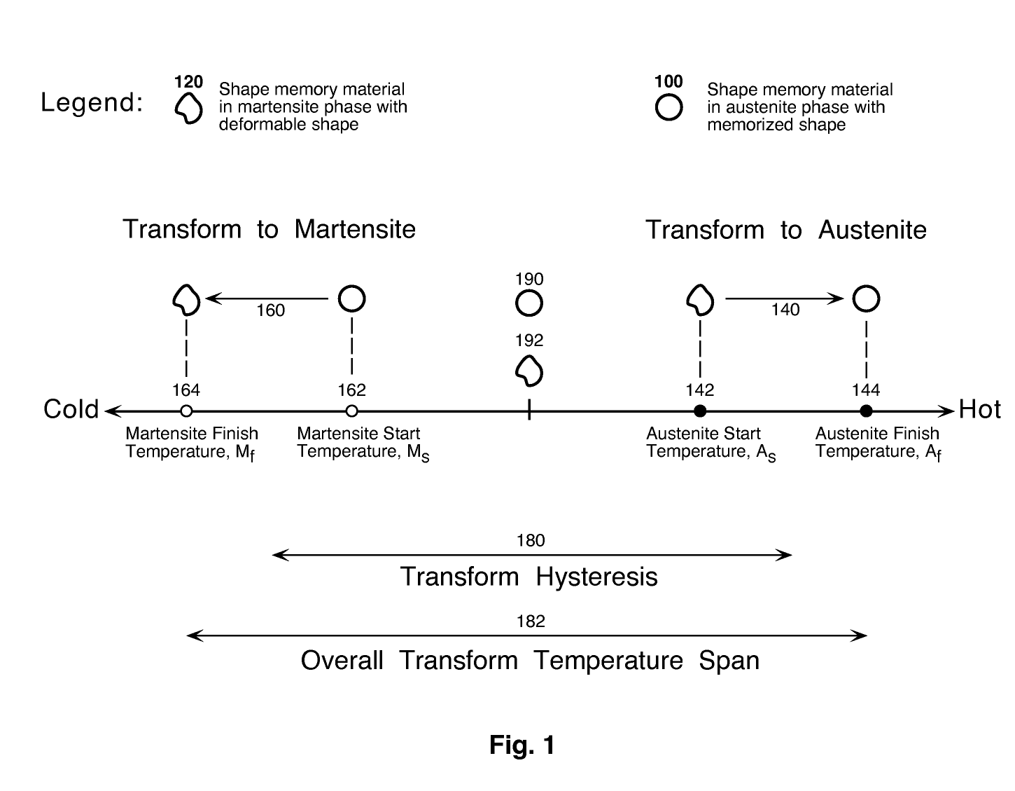

FIG. 1 is a temperature-induced shape memory phase change diagram showing the behavior of “one-way” shape memory material. Shape memory materials exist in two crystallographic structures: austenite (icon 100) and martensite (icon 120). The austenite phase is characterized by stiffness and superelastic properties. The martensite phase is soft and malleable. The shape of an austenite object is referred to as the “memorized shape.” An object in the austenite phase can be transformed into martensite by cooling. As soft martensite, the object can then be deformed. This martensite object can be transformed back into austenite by heating. Upon this phase conversion, the object's shape will return (with some force) to the “memorized shape.” The transformation from a defined austenite shape to an undefined martensite shape is called one-way shape memory.

The word “transform” as used herein shall refer to a phase change to or from a phase with a memorized shape. A transform temperature band is the difference in temperature from when a transform begins and when it ends. The martensite to austenite transform 140 occurs over a range of temperatures from Aₛ (austenite start) 142 to A_{f} (austenite finish) 144. Similarly, the austenite to martensite transform 160 occurs over a range of temperatures from Mₛ (martensite start) 162 to M_{f} (martensite finish) 164. The austenite transform and martensite transform occur within different transform temperature bands. This phenomenon is called transformation hysteresis 180, which is the temperature spread between an object that is 50% transformed to austenite upon heating and an object that is 50% transformed back to martensite upon cooling. The overall transform temperature span 182 is the temperature range one needs to transform an object between 100% martensite and 100% austenite. For nitinol, the overall transform temperature span is approximately 50° C. An important characteristic of shape memory materials is that an object can either be in its austenite phase 190 or martensite phase 192 at a temperature between the transform temperature bands depending upon its history of heating and cooling.

With nitinol, the transformation temperatures 142, 144, 162, and 164 are determined by the Ni to Ti atomic ratio and the metallurgical processing of the nitinol after alloy formation. Nitinol's austenite memorized shape is configured by metallurgical processing when the material is in its austenite phase.

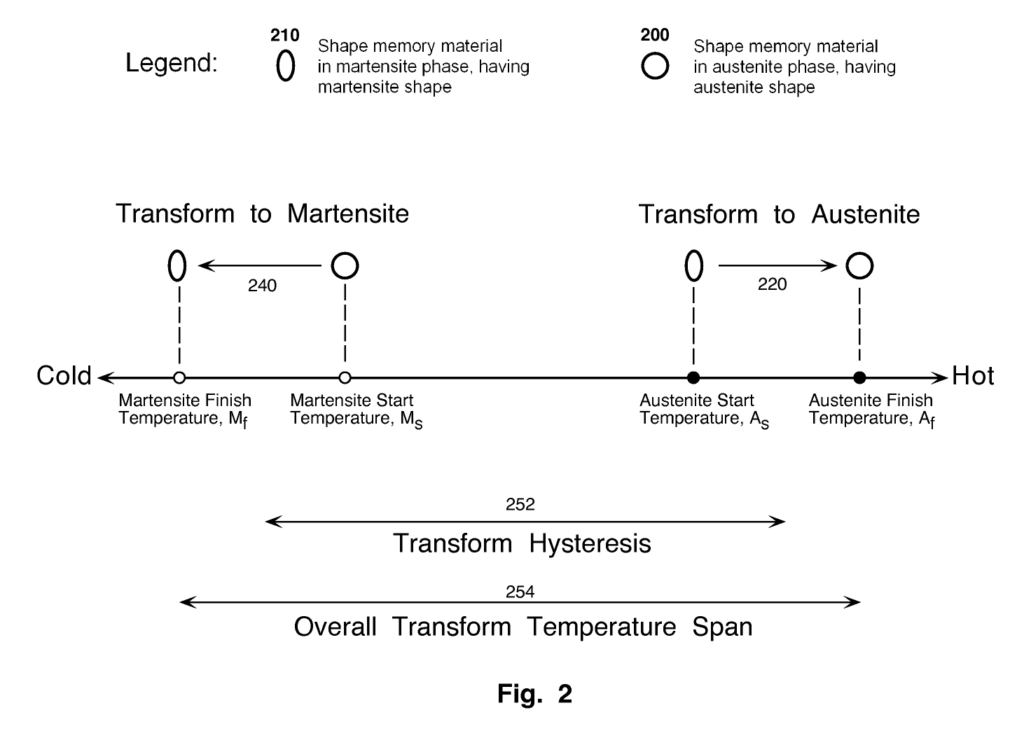

FIG. 2 is a temperature-induced shape memory phase change diagram for shape memory materials that exhibit two-way shape memory. Most shape memory materials that exhibit one-way shape memory can be trained to exhibit two-way shape memory. These materials exist in two crystallographic structures: austenite (icon 200) and martensite (icon 210). Objects fabricated from two-way shape memory materials will have two unique shapes depending on the phase. An austenite object is referred to as having the “austenite shape”. The shape of a martensite object is referred to as the “martensite shape”. Both shapes are firm and distinct. Hence both are “memorized” shapes. The temperature transforms 220 and 240 toggle the shape memory material between the phases and result in shape changes. Transform hysteresis 252 and overall transform temperature span 254 have a similar meaning as for one-way shape memory materials.

### Helical Spring Shape Memory Actuators

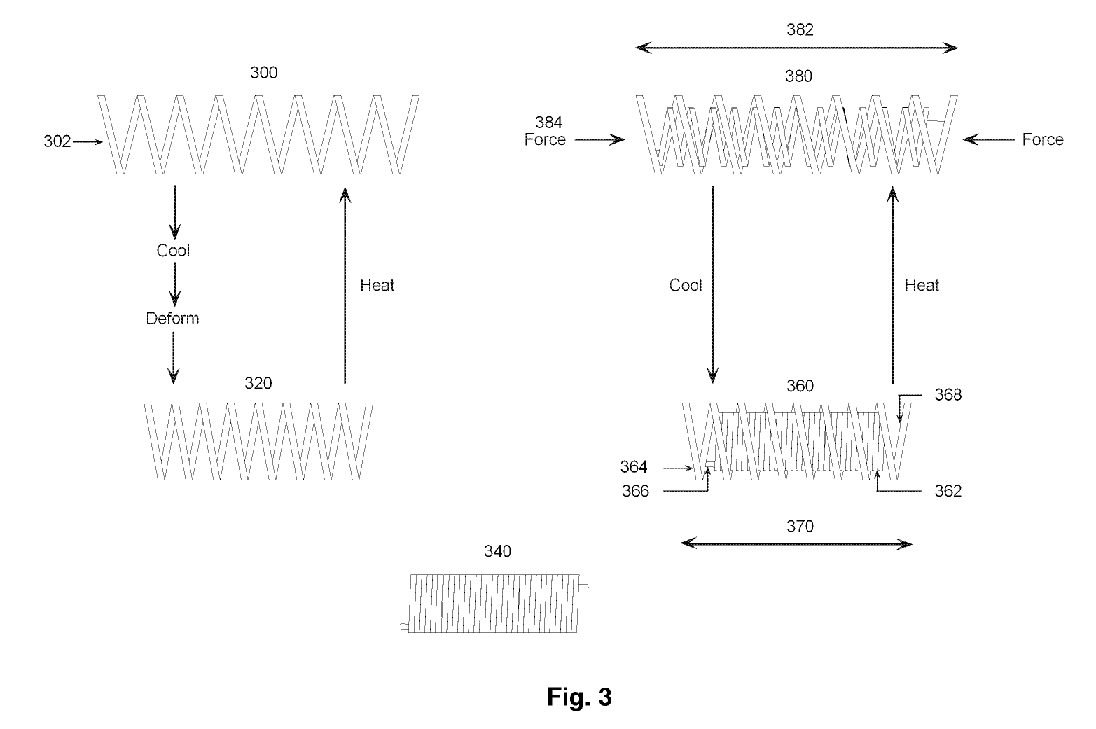

FIG. 3 illustrates how a spring made of one-way shape memory alloy can be combined with a spring made of non-shape memory material to form a temperature sensitive actuator called a helical spring shape memory actuator. Item 300 is a helical nitinol spring in its austenite phase and hence memorized shape. Within the helix is an open cylindrical space 302. The spring acts like a normal spring as long as the temperature is above the martensite start temperature. If the nitinol spring is cooled and transformed to martensite, however, it can be compressed or deformed 320 with relatively low force. It will remain in this compressed state if the compression force is released. If the cooled martensite nitinol spring is reheated back to its austenite phase its memorized shape 300 is restored.

Item 340 is a bias spring made from a non-shape memory spring material, such as steel, brass, or aluminum. It can also be made of non metals such as plastics.

When the two springs are combined and operatively engaged at their ends, a helical spring shape memory actuator is formed. The actuator will expand and contract as its temperature is raised and lowered such that the shape memory material therein toggles between its austenite and martensite phase.

Items 360 and 380 show the details of a helical spring shape memory actuator in its martensite and austenite phase respectively. The actuator comprises a bias spring 362 placed within the open cylindrical space of a nitinol spring 364. The two springs are attached together at their ends 366 and 368. If the nitinol spring is in its compressible martensitic phase, the bias spring dominates and the actuator's overall length 370 is compressed. If the actuator is heated (Item 380) above the nitinol spring's austenite transform temperature, then the nitinol spring will expand to its memorized shape and the actuator length 382 will increase. In this austenite transform, the force arising from nitinol spring seeking to attain its memorized shape will dominate the counter-force from the bias spring.

If the austenite actuator is cooled to below its martensite finish temperature, the nitinol spring contained therein will be transformed to martensite. The nitinol spring loses its stiffness, becomes malleable and can no longer dominate the bias spring. Therefore the actuator seeks to attain its martensite length 370. If the actuator is physically blocked from contracting to this length, then a “1 W actuator contraction force” 384 will be imposed on whatever is blocking it. Hence shape memory actuators can be used as temperature driven clamps.

An alternative configuration for an actuator is to have the austenitic length of the nitinol spring be shorter than the length of the bias spring. The bias spring would be compressed to be joined with this austenitic nitinol spring. If such an actuator is then cooled below its martensite transform temperature, it will exert a force on the constraining members in the opposite direction of the arrows in item 384. This force will be referred to as “1 W actuator expansion force”. Hence shape memory actuators can be designed as clamps that either push or pull.

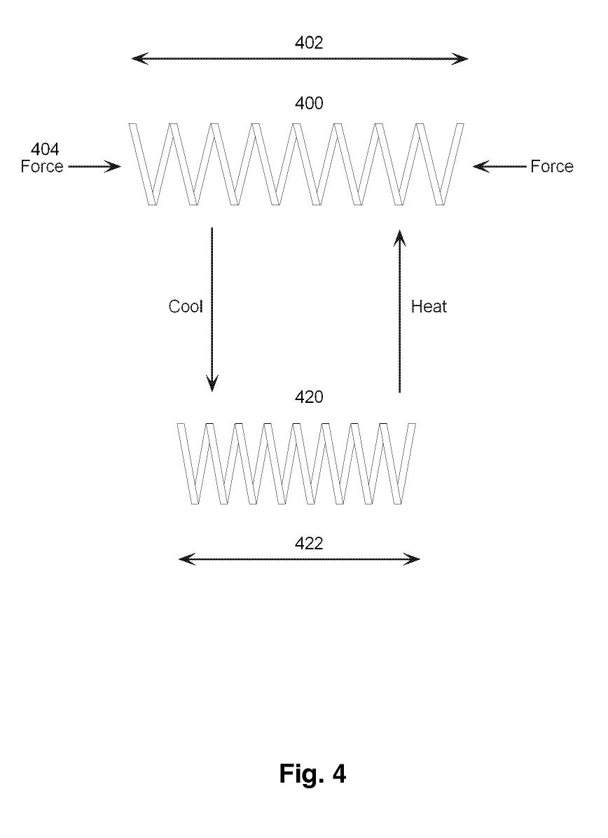

FIG. 4 illustrates how two-way shape memory materials can be used to fabricate helical spring actuators. Two-way nitinol spring 400 is in its austenite phase and has an expanded length 402. This is its austenite shape. Its martensite shape 420 is a spring having a shorter length 422. If the austenitic two-way nitinol spring is cooled to below its martensite finish temperature, it will morphologically change to its martensite shape. If during this martensite transform, the actuator is constrained from achieving the short length 422, then a force 404 will be exerted on the constraining members. This force will be referred to as the “2 W actuator contraction force”. If the length of the austenite two-way spring is shorter than its martensite shape, the martensite transform induced force on constraining members is in the opposite direction of arrows 404. This force will be referred to as “2 W actuator expansion force.”

### Cylindrical Shape Memory Actuators

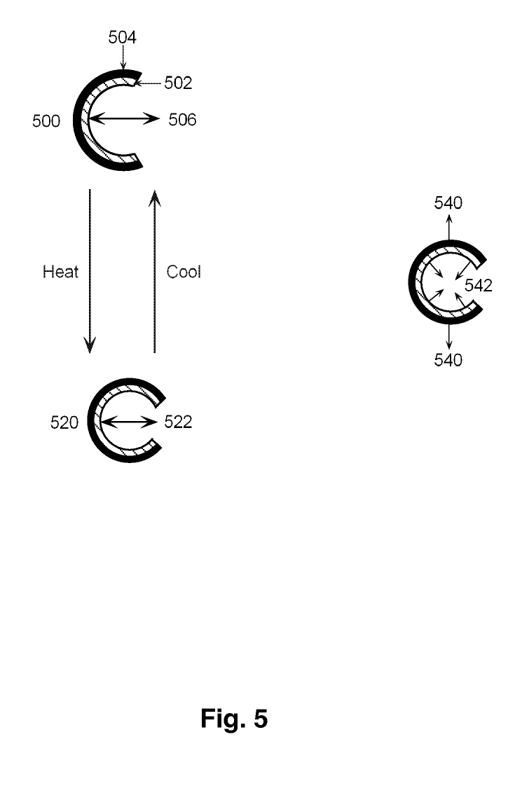

FIG. 5 shows a cross sectional view of a cylindrical shape memory actuator 500. The cylindrical shape memory actuator comprises a one-way shape memory roll pin component 502 operatively engaged (e.g. joined) with an ordinary metal bias roll pin 504. The bias roll pin has spring-like properties. The actuator has a characteristic open diameter of 506 when the shape memory roll pin component is in its martensite phase. If the actuator is warmed to above its austenite finish temperature, the shape memory roll pin component will transform to its austenite phase and hence its memorized shape 520. This shape is a roll pin with characteristic diameter 522. In this austenite transform, the force arising from nitinol roll pin seeking to attain its memorized shape will dominate the counter-force from the bias roll pin. If the austenite roll pin is cooled to below its martensite finish temperature, it will seek to return to its martensitic characteristic diameter 506. If the actuator is constrained from achieving this characteristic diameter, then a pressure force 540 will be exerted on the constraining members.

Roll pin actuators can be made such that the martensite transform causes its shape to shrink. This configuration can be described by switching the words “heat” and “cool” in FIG. 5. Forces arising from a martensite transform would be clamping force 542.

### Cryogenic Container Closing Device

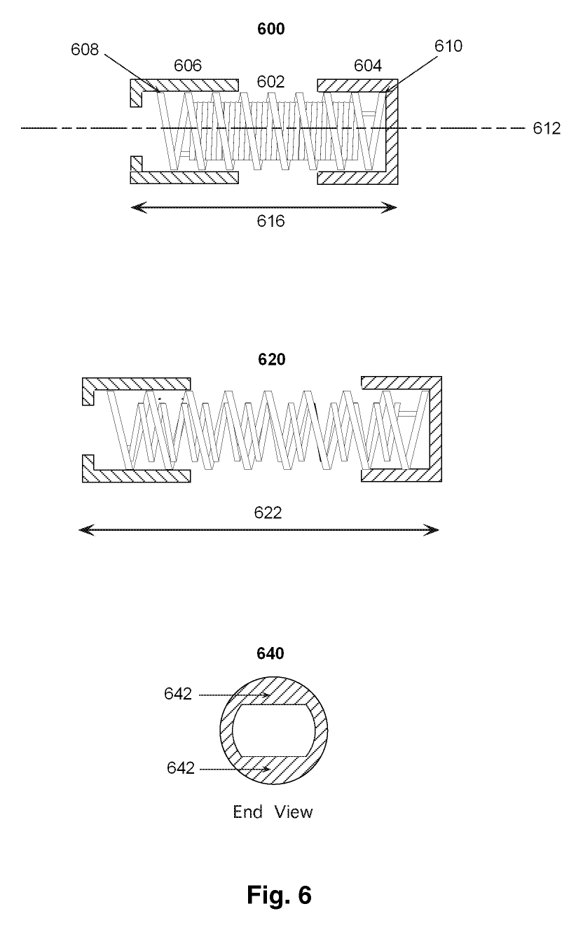

FIG. 6 illustrates a cryogenic container closing device that relies on the clamping abilities of helical spring shape memory actuators. A closing device 600 comprises a helical spring shape memory actuator 602, an end cap 604 and a latching sleeve 606. These components are attached at 608 and 610. The closing device is shown with the actuator in its relatively closed 616 martensitic phase. The closing device is free to expand and contract along axis 612 with temperature changes above and below its martensite and austenite transform temperatures. Item 620 shows the closing device after it has been warmed to its expanded 622 austenitic phase.

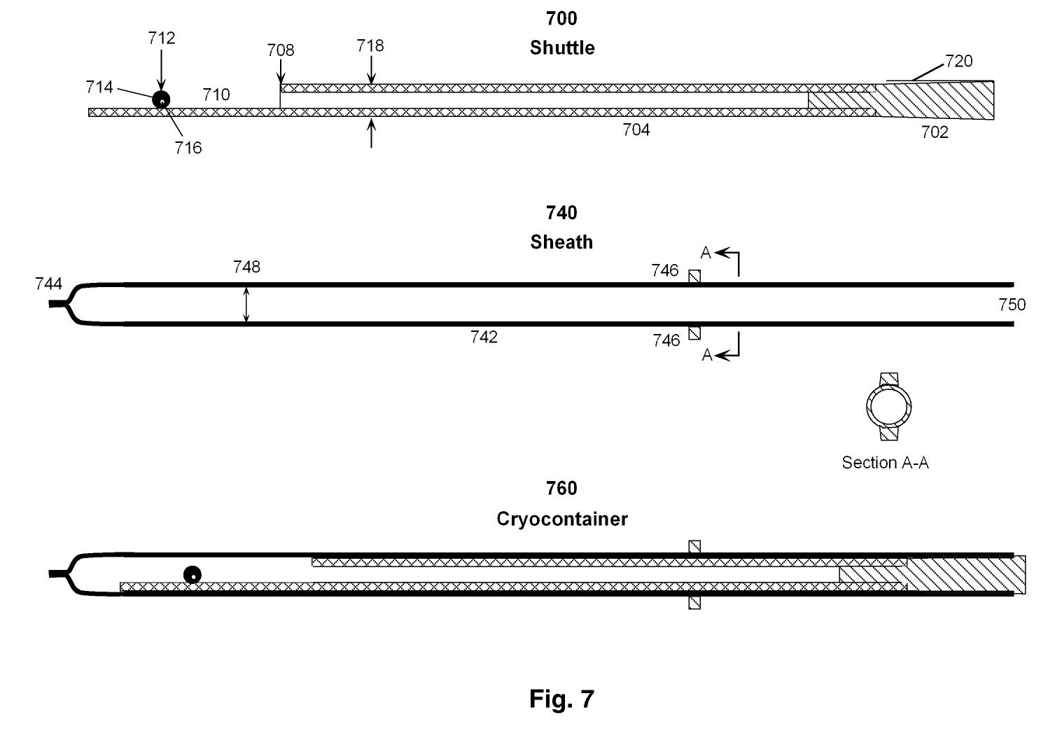

An end view 640 of the latching sleeve shows latches 642 used to engage locking tangs (Item 746, FIG. 7) on a cryogenic container (Item 760, FIG. 7).

FIG. 7 illustrates longitudinal sections of generally tubular elements of an exemplary cryocontainer that can be closed and sealed by the closing device. The cryocontainer can be used for vitrification of a biological specimen and is described in more detail in copending U.S. patent application Ser. No. 12/267,708.

The cryocontainer comprises a shuttle 700 and sheath 740.

The shuttle comprises a stopper 702 and tube 704 with notch 708 cut in the end to provide a channel 710 for holding a biological specimen 712. The biological specimen is to be vitrified by rapid chilling to cryogenic (e.g. −80° C. and below) temperatures. The biological specimen may comprise vitrification media 714 and one or more cells, 716 that are to be cryopreserved. The diameter 718 of the shuttle is larger than the diameter of the biological specimen. Typical specimens have volume of 0.5 micro liters with a corresponding diameter of about 1 mm. A suitable diameter for the shuttle, therefore, is 2 mm.

The stopper has a modest taper 720 so it will seal with the sheath when the shuttle is loaded therein. Suitable taper angles may be within the range of about 1° to 15° measured with respect to the longitudinal axis of the stopper. The stopper should be made of a material that is resilient at cryogenic temperatures to help maintain the seal. Teflon is a suitable material.

The sheath 740 comprises a tubular body 742 and locking tangs 746 on the exterior. One end of the sheath is sealed 744. A suitable inner diameter 748 for the sheath is 2.1-2.5 mm when the diameter of the shuttle is 2 mm.

Item 760 is an assembled cryocontainer ready for the installation of the closing device. The shuttle containing the biological specimen is advanced into the open end 750 of the sheath until the stopper engages the sheath. Only hand force is required to seat the stopper.

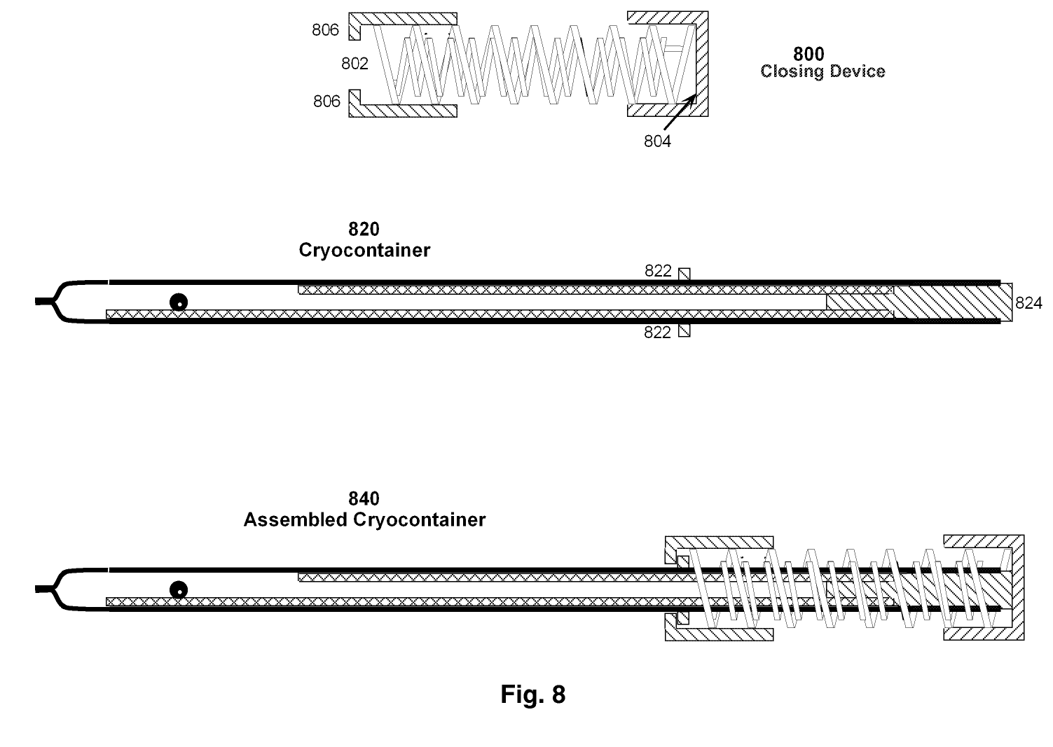

FIG. 8 illustrates how a closing device 800 can be combined with a cryocontainer 720 to form an aseptic seal for long term cryogenic storage of a biological specimen. The open end 802 of the closing device in its high temperature expanded austenitic phase is placed over the stopper end 824 of the cryocontainer and twisted so that the latches 806 engage the locking tangs 822 on the cryocontainer. Assembled cryocontainer 840 is then formed. The end cap 804 of the closing device rests against the stopper 824.

The closing device may then be grasped by chilled forceps. This causes the closing device to transform to its martensite phase and attempt to contract. This in turn causes a 1 W contraction force to be placed on the stopper, thus sealing the device. The entire assembly is then immersed in LN2 to effect vitrification. It remains sealed and can be placed on long term cryogenic storage. As long as the device remains below its austenitic start temperature, the clamping force is maintained and the cryocontainer remains sealed.

When it is necessary to recover the biological specimen, the cryocontainer is transferred from cryogenic storage to a warm water bath (e.g. 37° C., body temperature). The water bath is warm enough to transform the nitinol actuator to its austenite phase and the closing device expands. It can then be removed, the stopper unplugged and the shuttle taken out of the sheath for recovery of the biological specimen.

### Suitable Materials for Biological Specimens

Biological specimens, such as human reproductive cells, may come in contact with various components of a cryocontainer. Human reproductive cells are negatively sensitive to certain materials. Materials that do not cause such a reaction are called “non-embryotoxic.” Thus, suitable materials for the end cap, latching sleeve, shuttle and sheath include non-embryotoxic materials suitable for cryogenic service. lonomer resins such a Surlyn 8921 are suitable. Our tests have shown that nitinol is non-embryotoxic according to standardized tests and therefore is suitable as well. Nitinol can be used at cryogenic temperatures.

### Alternative Closure Device

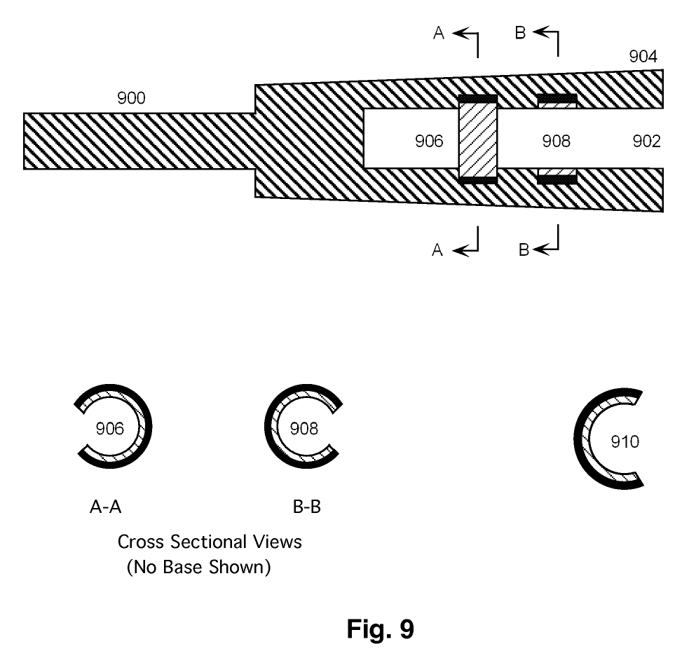

FIG. 9 illustrates an alternative method to use shape memory actuators to seal a vitrification cryocontainer. Stopper 900 can be incorporated in a shuttle for which an external closing device is not required to seal. The stopper comprises two roll pin actuators 906, 908 co-molded within the base 904. Opening 902 exposes the actuators to the environment. The two actuators are offset (see cross sectional views A-A and B-B) 180 degrees to provide relatively uniform sealing pressure. At the loading temperature (e.g. room temperature), the actuators are in their austenite phase and relatively collapsed or contracted. Loading and seating the shuttle in the sheath is similar to as described above. The stopper may then be grasped by chilled forceps at a point close to the actuators. This causes the actuators to transform to their martensite phase and attempt to expand to their martensitic shape 910. This presses the stopper against the inner wall of the sheath creating a seal. The entire assembly is then immersed in LN2 to effect vitrification. As long as the actuators remains below their austenitic start temperature, the pressure force is maintained and the cryocontainer remains sealed.

When it is necessary to recover the biological specimen, the cryocontainer is transferred from cryogenic storage to a warm water bath (e.g. 37° C., body temperature). The water bath is warm enough to transform the actuators to their austenite phase and contract, thus loosening the stopper. The stopper can then be removed and the shuttle taken out of the sheath for recovery of the biological specimen.

### Suitable Nitinol Grades for Shape Memory Actuator used in Vitrification Applications

In vitrification, a biological specimen is normally loaded at room temperature (about 20° C.). Vitrification and storage is typically done in liquid nitrogen (about −196° C.).

Warming is typically done in a body temperature water bath (about 37° C.). Therefore, the attributes of a suitable nitinol grade are:
1. In its austenite phase at room temperature after being first warmed to above its austenite finish temperature;
2. It's capable of returning to its memorized shape by 37° C.;
3. It has a martensite finish temperature no colder than the chilling cryogen.

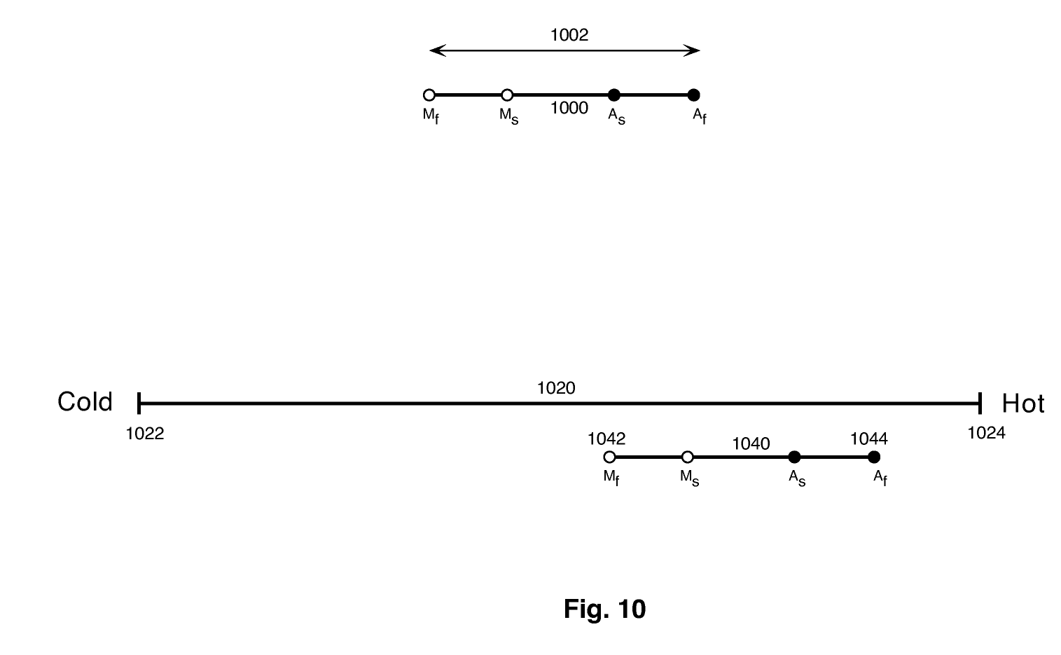

FIG. 10 shows a typical nitinol transform temperature profile 1000. The overall transform temperature span 1002 is typically about 50° C. The temperature scale 1020 identifies a minimum temperature 1022, which is the lowest possible martensite finish temperature that satisfies (3) above. For ordinary nitinol, this limit is about −100° C., the minimum temperature that transforms will occur at. A nitinol alloy can be modified with the addition of iron or chromium that will reduce the minimum temperature to the temperature of LN2 (−196° C.). The maximum temperature 1024, needs to satisfy (2) above. The memorized shape of nitinol is substantially restored within a few degrees less than the austenite finish temperature. We can set the maximum temperature to 40° C. Therefore, there is a surprisingly wide range of nitinol alloys are suitable for this application. The nitinol transform temperature profile 1040 can be placed anywhere on the temperature scale so long as the ends (1042 and 1044) do not exceed the respective minimum and maximum temperature limits.

Other remarkable observations about the application of nitinol to closing devices for vitrification:
1. Unlike other shape memory actuation applications, the wide temperature span needed to seal and unseal the cryocontainer occurs naturally in the vitrification process.
2. Transform hysteresis confines transform motion to the temperature transform bands. “Dead zones” are quite common in working with shape memory alloys. This limitation does not affect vitrification closing devices.
3. Within a temperature transform band, the motion is always in one direction. This limitation does not affect vitrification cryocontainers.

A preferred nitinol alloy is one with an austenite finish temperature in the range of 0° C. to 10° C. A closing device with this alloy will reliably stay open at room temperature and will easily open when the specimen is warmed.

### Alternative Embodiments

This disclosure provides several embodiments that use shape memory materials to seal and unseal a vitrification cryocontainer. It creates forceful motion from the toggling of shape memory materials between austenite and martensite phases and, in some cases, in conjunction with bias springs. In turn, this forceful motion is applied to sealing surfaces to make (during chilling) or break (during warming) a seal. We have described how an actuator that exhibits 1 W actuator contraction force can be utilized in this invention. Also, we have described how a roll pin actuator that exhibits pressure force can be utilized. Other possible ways to use shape memory materials to fabricate a suitable seal are:
1. A closing device with a 2 W actuator, consisting of a nitinol spring that creates 2 W contraction force. This method uses the cryocontainer design described above.
2. An alternative design for the closing device can use the 1 W or 2 W expansion forces as its closing force.
3. An alternative design for the closing device can use clamping forces derived from a roll pin actuator.

### Conclusion

While the disclosure has been described with reference to one or more different exemplary embodiments, it will be understood by those skilled in the art that various changes may be made and equivalents may be substituted for elements thereof without departing from the scope of the disclosure. In addition, many modifications may be made to adapt to a particular situation without departing from the essential scope or teachings thereof. Therefore, it is intended that the disclosure not be limited to the particular embodiment disclosed as the best mode contemplated for carrying out this invention.

1. A closing device for a cryocontainer comprising a shape memory actuator, said shape memory actuator comprising a shape memory material, wherein said actuator is biased to automatically provide a force to seal said cryocontainer when said closing device is cooled to a cryogenic temperature.

2. The closing device of claim 1 wherein said actuator comprises a means for attaching said closing device to said cryocontainer.

3. The closing device of claim 1 wherein said shape memory material is selected such that said sealing force is relieved when said closing device is warmed to body temperature thereby unsealing said cryocontainer.

4. The closing device of claim 1 wherein said sealing force is due to a martensite transform of said shape memory material and said relief of said force is due to an austenite transform of said shape memory material.

5. The closing device of claim 1 wherein said shape memory material is a nitinol alloy.

6. The closing device of claim 5 wherein said nitinol alloy comprises iron or chromium such that the martensite finish temperature of said alloy is less than about −100° C.

7. The closing device of claim 5 wherein said nitinol alloy is a one-way shape memory alloy.

8. The closing device of claim 7 wherein said shape memory actuator comprises a bias spring located coaxially with a nitinol spring and wherein the ends of each of said springs are operatively engaged.

9. The closing device of claim 8 wherein said force is either an expansion force or a contraction force.

10. The closing device of claim 7 wherein said shape memory actuator comprises a bias roll pin operatively engaged with a nitinol roll pin.

11. The closing device of claim 10 wherein said force is either a clamping force or a pressure force.

12. The closing device of claim 5 wherein said nitinol is a two-way shape memory alloy.

13. The closing device of claim 12 wherein said shape memory actuator comprises a helical spring

14. The closing device of claim 13 wherein said force is either an expansion force or a contraction force.

15. The closing device of claim 12 wherein said shape memory actuator comprises a roll pin.

16. The closing device of claim 15 wherein the said force is either a clamping force or a pressure force.

17. A cryocontainer for vitrifying and storing a biological specimen at a cryogenic temperature, said cryocontainer comprising:
a. a shuttle that is configured to receive a tapered stopper within an end portion of the shuttle; and
b. a sheath comprising a open end dimensioned to receive and seal against said stopper,
wherein said stopper is sufficiently resilient at said cryogenic temperature to form an aseptic seal with said sheath.

18. The cryocontainer of claim 17 further comprising a shape memory closing device, said shape memory closing device configured to deliver a force to seal said stopper to said sheath when said closing device is at said cryogenic temperature.

19. The cryocontainer of claim 18 wherein said sheath comprises at least one locking tang and said closure device comprises at least one latch whereby a first portion of said closing device is configured to operatively engage with said sheath by engaging said latch with said tang and a second portion of the closing device is configured to engage the stopper, wherein the closing device is configured to deform when approaching said cryogenic temperature to establish said aseptic seal.

20. The cryocontainer of claim 19, wherein said deformation of said closing device urges the stopper into the shuttle.

21. The cryocontainer of claim 20, wherein said deformation of said closing device is relieved when the temperature of said closing device rises above said cryogenic temperature.

22. The cryocontainer of claim 18, wherein said shape memory closing device is fixed to said stopper and automatically transforms between a first contracted configuration when said closing device is substantially above said cryogenic temperature, and said closing device transforms to an expanded orientation when said closing device is at said cryogenic temperature.

23. The cryocontainer of claim 22, wherein said stopper is configured to compress against an inner wall portion of said shuttle when said closing device is in said expanded orientation and said stopper is configured to retract away from said inner wall portion of said shuttle when said closing device is in said contracted configuration.
